(19) United States
(12) Patent Application Publication
(10) Pub. No.: US 2012/0041344 A1
(43) Pub. Date: Feb. 16, 2012

(54) APPARATUS AND METHOD FOR ESTIMATING BODY FAT MASS

(76) Inventor: Carl-Erik Flodmark, Malmo (SE)

(21) Appl. No.: 13/257,522

(22) PCT Filed: Mar. 18, 2010
(86) PCT No.: PCT/SE10/50306

§ 371 Date: Oct. 14, 2011

Related U.S. Application Data
(60) Provisional application No. 61/161,940, filed on Mar. 20, 2009.

(30) Foreign Application Priority Data
Mar. 20, 2009 (SE) 0900360.9
Nov. 13, 2009 (SE) 0950859-9

(51) Int. Cl. A61B 5/103

(52) U.S. Cl. 600/587

(57) ABSTRACT

An apparatus (100) for measuring body fat mass and degree of obesity of a person (P) is provided. According to one aspect, the apparatus comprises a second bar (102) optionally configured to be placed on a horizontal surface, and a first bar (101) configured to be movable along and being perpendicular to said second bar (102) and an optional control unit (111). A first connecting means (105) connects said first bar (101) to said second bar (102). A light emitting device (104) is connected to said first sliding member (103) and is configured to emit light in a direction perpendicular to the plane defined by the first and second bar (101, 102). According to another aspect, the apparatus comprises a hanging bar (129) configured to be placed in front of said person (P), a supporting bar (128) configured to provide support for the hanging bar (129), a second sliding member (133) being slidingly connected to said hanging bar (129), at least one light emitting device (104) configured to emit a sheet of light (114) directed toward the body of said person (P) whereby at least one line (L4) is projected onto the body of said person (P), at least one camera (134) configured to detect line (L4), wherein at least one of either said at least one light emitting device (104) or said at least one camera (134) is connected to said second sliding member (133). A system and a method thereof is also provided.

### TECHNICAL FIELD

The present invention relates to an apparatus, a system and methods for the determination of two-dimensional coordinates of a person, such as a child or adolescent, reflective of e.g. the body fat mass and degree of obesity of the same.

### BACKGROUND

Obesity is a significant and growing health problem, well known and prevalent in the western world. To the individual, being overweight or obese is a major negative physical, medical and very often psychological, factor. To the society, obesity amongst the citizens constitutes a large economic factor, primarily due to the cost related to dependant secondary diseases and conditions, such as diabetes and cardiovascular malfunction.

Overweight children and adolescents have a significantly higher risk of becoming overweight or obese adults in comparison to children and adolescents with a normal body fat mass, respectively. Hence, it is of great importance to provide methods and means for measurement and quantification of body fat mass, especially in the groups of children and adolescents.

The absolute figures, i.e. metrics for the degree of obesity, serve the physician or parents as a check on whether or not the child or adolescent is in a risk zone by comparison with statistic data. He or she may furthermore compare these metrics with historic individual data to assess the improvement with regard to decrease in body fat mass over time. Based on the outcome of these comparisons, the physician, the parents, or anyone else being responsible, may take suitable measures with regard to style of life, physical activity and healthful eating.

In addition to being highly valuable for the classification and tracking of body fat mass of persons where potential (future) obesity is of concern, these metrics may also be used for the analog classification and tracking of persons where abnormally low body fat mass is of concern, such as for persons with anorexia nervosa.

A well known method to measure and indirectly quantify a persons body fat mass is to measure that persons weight and length on a scale, and there from calculating the corresponding body mass index (BMI) reflective of health risks. Although the measurement of a persons BMI is a relevant metric for body fat mass in the adult, the same is not true for a child or adolescent as such a person is in a growing state and, accordingly, the BMI naturally changes over time and is thus only partly dependant of the body fat mass.

EP 1,269,917 A1 describes the measurement of skinfold thickness, related to body fat mass, of a living body by essentially placing a protruding part onto an umbilical region of the living body and irradiating the same with the light from a light source, and detecting the diffusely reflected light with a photodetector. Disadvantages of this methodology include the discomfort caused to the person being measured by e.g. the protruding part, which by necessity is temporarily deforming the persons skin, and by the holding means, which is provided with e.g. a string or a belt wrapped around the persons body.

EP 1,629,772 A1 discloses a child body composition meter in the form of a platform comprising a bioelectrical impedance measuring section, a weight measuring section and a programmed microcomputer. The data generated by the former two being related to body fat mass. Disadvantages of this platform include the inability to get quantitative data related to the local body fat mass at different parts of the body and the advanced electronics needed to measure, transform and present the generated data. Furthermore, the child being measured needs to actively and potentially uncomfortably stand on the platform with naked feet in order to allow for e.g. an electric current, necessary for the bioelectrical impedance measurement, to be passed through the body.

EP 1,583,019 A2 discloses a system for and a method of managing growth and development of a child. The system includes, similar to EP 1,629,772 A1, a biological information measuring module which is based on the principles of e.g. the measurement of bioelectrical impedance, related to body fat mass. The disadvantages of the system disclosed in EP 1,583,019 A2 include the disadvantages of the child body composition meter disclosed in EP 1,629,772 A1.

EP 1,645,223 A2 describes an apparatus and a method of measuring fat thickness in a target body part of a person based on the principles of absorption and reflection of near infrared rays. Disadvantages of this method include the necessity of a calculator, and of complicated analysis and transformation of the generated raw-data, in order to retrieve metrics related to body fat mass. Furthermore, the long term health effects of penetrating electromagnetic radiation is widely debated and not yet fully understood.

EP 2,016,895 A1 describes a body composition measuring apparatus for estimating a body composition index. The apparatus includes an abdominal width measurer which is employing reflection-type contactless distance measuring sensors arranged on a frame to measure the abdominal width of a person inside the same. These sensors are measuring a gap distance between a position of a sensor and a position of a point to be measured. Each sensor includes a light emitter for emitting light, and a light receiver for receiving the light reflected from e.g. a person's skin. Disadvantages of reflection-type distance measuring sensors of e.g. this method include potential range detection errors arising from energy emanations from other sensors and light energy scattering. Furthermore, this type of sensors depend on satisfactory reflection of light from, in this application, human skin, which might be poor if e.g. the emitted light is entering at an angle not being perpendicular to the skin surface, or if the skin is of a type with poor ability to reflect light, such as dark skin.

Hence, there is room for improvements, namely a new technique, for measuring or estimating body fat mass and degree of obesity of a person, which technique allows for accurate measurements with limited measurement errors and limited discomfort and health risks to the person, while simultaneously limiting the need for advanced electronics and for passing current through the body of the person, or exposing the same to penetrating electromagnetic radiation.

### SUMMARY

It is an object of the present invention, considering the disadvantages mentioned above, to provide a simple and mobile apparatus and system to be used for the collection of relevant data based on accurate measurements with limited measurement errors being reflective of a person's body proportions in a cost effective way that causes minimal discomfort and risk to the person.

It is another object of the present invention, to provide a method for the use of the apparatus and system of the invention for the collection of two-dimensional coordinates or equivalent data, reflective of a person's body proportions and the body fat mass of the same.

These and other objects, which will appear from the following description, have now been achieved by an apparatus and system according to the present invention which comprises a first and a second bar, said first bar being configured to be movable along said second bar and said first bar being perpendicular to said second bar; first connecting means for connecting said first bar to said second bar; and a light emitting device configured to emit light in a direction perpendicular to the plane defined by the first and second bars and towards said person.

Further features of the invention and its embodiments are set forth in the appended claims.

### BRIEF DESCRIPTION OF THE DRAWINGS

These and other aspects, features and advantages of which the invention is capable of will be apparent and elucidated from the following description of non-limiting embodiments of the present invention, reference being made to the accompanying drawings, in which

FIG. 1 is a schematic view of an apparatus, placed with a first bar in the vertical direction and with a second bar in the horizontal direction and with a person P placed horizontally with a sagittal plane of the body facing the apparatus, according to an embodiment of the present invention;

FIG. 2 illustrates an embodiment of the present invention, showing the left side, i.e. sagittal view, of the body of a person P lying on the back on a flat horizontal surface, as seen from the z-direction of the apparatus;

FIG. 3 is a schematic view of an apparatus comprising an integrated bench, an array of light beams illuminating light sensing means positioned on a third bar and a sheet of light illuminating an extension unit provided with light sensing means, according to an embodiment of the present invention;

FIG. 4 is a schematic view of an apparatus comprising a connecting bar provided with a light emitting device constituted by a plurality of lasers that emits a plurality of beams of light toward an integrated bench, according to an embodiment of the present invention;

FIG. 5 is a schematic view of an apparatus comprising a bed plate placed on a vertical bench and onto which a person may be placed and onto which a supporting bar with a shape of an “inverted L” is fastened, a hanging bar fastened onto the supporting bar and extending in the x-direction above the bed plate, a second sliding member slidingly engaged to the hanging bar and onto which a camera and a light emitting device is attached, the light emitting device is emitting a sheet of light which is giving rice to a reflection in the form of a line on the bed plate and on the bench below it, a control unit in the form of a portable PC which is connected to the light emitting device and the camera to provide output to and input from these, according to an embodiment of the present invention; and

FIG. 6 is a schematic sectional view from above a person lying down horizontally on a flat surface being exposed to a sheet of light emitted from a light emitting device from right above whereby a line is projected partly on the surface and partly on the exposed part of the body of the person.

### DETAILED DESCRIPTION OF THE EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

As well known, the x-y-z coordinate system, wherein the constituting x-, y- and z-axis are all perpendicular to each other, is commonly used to describe e.g. points, lines and planes in three dimensions. In the description, embodiments and claims of the present invention, the x-y-z coordinate system is used with reference to the apparatus 100, such that the x-axis of the x-y-z coordinate system is always parallel with the direction of second bar 102, the y-axis of the same is always parallel with the first bar 101, and the corresponding z-axis is always perpendicular to the plane defined by the first and second bars 101, 102, unless otherwise indicated. Hence, for example, with “x-direction” is to be understood a direction in space parallel to, or along, the elongation of the second bar 102, and with “x-position” is to be understood a point in a plane transverse to the same position on the second bar 102 or the relative position on the second bar 102, unless otherwise indicated.

In the description, embodiments and claims of the present invention, the well known terms “horizontal” and “vertical”, or derived forms thereof, are used to describe e.g. points, lines and planes in the room, such that the vertical direction is always parallel to the direction of the force of gravity, unless otherwise indicated.

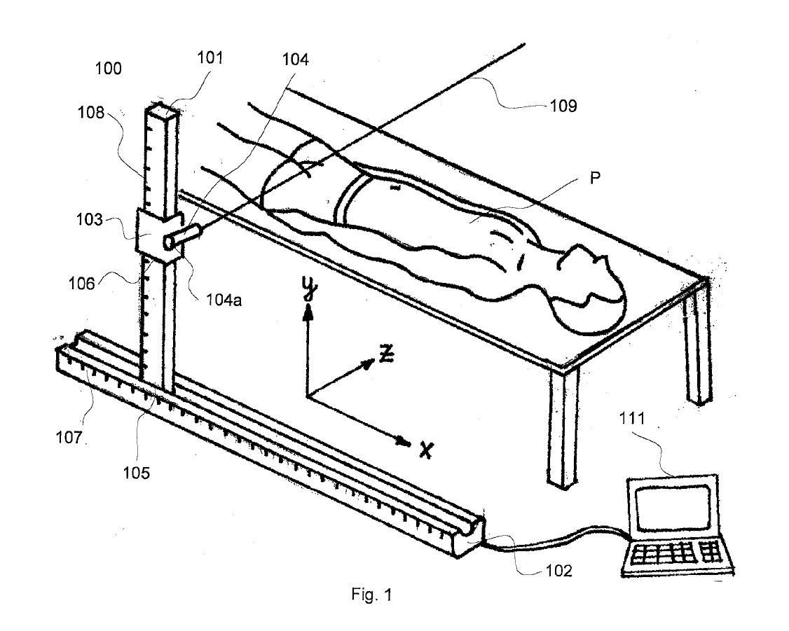

Embodiments of the present invention will now be described below with reference to FIGS. 1 to 6.

According to an embodiment of the invention as illustrated in FIG. 1, there is provided an apparatus 100 and a method for the measurement and determination of two-dimensional coordinates of a person P, including essentially a straight first bar 101, a straight second bar 102 and a light emitting device 104.

The first bar 101 is connected to the second bar 102 by first connecting means 105 to allow a user to move the first bar 101 to a position in the x-direction on the second bar 102 as desired by the user and only limited by the effective length of the second bar 102. The angle between the first bar 101 and the second bar 102 is substantially 90° independent of the x-position of the former.

First bar 101 and second bar 102 may be made of the same or different materials, preferably selected from materials known in the art as constituents of bars, such as wood, plastic, aluminum or suitable composites. Even more preferred is a suitable material that is cheap, light and environmentally friendly in order to make the total cost of the apparatus 100 relatively low, the transportability of the apparatus 100 relatively high, and the apparatus 100 recyclable to a great extent, respectively.

Second bar 102 is preferably made of a heavier material, or has a greater total mass, than first bar 101 for the sake of stabilization of apparatus 100 when it is positioned with the first bar 101 and the second bar 102 oriented in the vertical and horizontal direction, respectively.

When the first bar 101 has been moved to a desired x-position, it remains in that position by first connecting means 105 of the connection between the first and second bars 101, 102 until a user moves it to another x-position as desired.

A first sliding member 103 is connected to the first bar 101 by second connecting means 106 to allow a user to move the first sliding member 103 to a position in the y-direction on the first bar 101 as desired by the user and only limited by the effective length of the first bar 101. When the first sliding member 103 has been moved to a desired y-position, it remains in that position by second connecting means 106 of the connection between the first bar 101 and the first sliding member 103 until a user moves it to another y-position as desired.

First and second connecting means 105, 106 include, for example, a tightening bolt, a natural friction element, or a fitting suitable for connecting a first bar to a second bar, and which fitting is well known to the person skilled in the art. One or both of first and second connecting means 105, 106 are preferably of a user-friendly type so that a user of the apparatus 100 intuitively understands their function and proper handling.

The x-position of the first bar 101 is equivalent to its, at that point in time, relative position on the second bar 102, which is determined by means for x-position determination 107, such as a visual reading of a scale positioned on the second bar 102. The means for x-position determination 107 may also be, as known in the art, an electric, magnetic, and/or mechanic sensor determination means, suitably triggered by a user, on the second bar 102.

The y-position of the first sliding member 103 is equivalent to its, at that point in time, relative position on the first bar 101, which is determined by means for y-position determination 108, such as a visual reading of a scale positioned on the first bar 101. The means for y-position determination 108 may also be, as known in the art, an electric, magnetic, and/or mechanic sensor determination means, suitably triggered by a user, on the first bar 101.

Visual reading of a scale positioned on the first or second bar 101, 102 is intuitive to a user and prevents otherwise common errors associated with more complicated systems for collection of data.

A light emitting device 104 is connected to the first sliding member 103 whereby it, at all times, has the same x-position as the first sliding member 103 and in such a way that the emitted beam of light 109 is substantially perpendicular, i.e. normal to, the plane defined by the first and second bars 101, 102, i.e. the plane defined by the y-axis and the x-axis, respectively. The emitted light may also be a sheet of light, traveling in a plane substantially perpendicular to the plane defined by the first and second bars 101, 102. The beam of light 109, or the center of the sheet of light, may have a direction within an error margin of, for example, 0 to 3°, relative an imaginary line perpendicular to the plane defined by the first and second bars 101, 102. Thus, the center of the beam of light 109, or the center of the corresponding sheet of light, has practically the same y- and x-coordinates along an axis (z) perpendicular to the plane defined by the y-axis and the x-axis.

Validation of the precision and accuracy of the beam of light 109, and thus the apparatus 100 in its setting, may be done simply by directing the same toward reference spots, for example on a wall, and comparing the corresponding x- and y-positions with accurate reference values. This way of validation is comparably simpler than the validation of many other instruments or systems used for the estimation of body fat mass.

Means 104a for connecting the light emitting device 104 to the first sliding member 103 include, for example, bolts or screws or suitable adhesive means.

The light emitting device 104 may also be connected to the first sliding member 103 by means 104a of a suitable gyrostabilizer known in the art, in order to e.g. stabilize the direction of the emitted beam of light 109 against e.g. temporal dislocations or vibrations of the apparatus 100.

In the case the direction of the beam of light 109 is, after triggered by the user as desired, periodically varying in one plane, such as in the y-plane or in the x-plane, an imaginary line drawn from the center of the resulting projection in the x-y-plane to the light emitting device 104, is practically parallel to the z-axis of the x-y-z coordinate system.

In order to prevent e.g. accidental tilting or dislocation of the apparatus 100, such as when the first bar 101 is placed in a vertical direction and the second bar 102 is placed on a horizontal surface, which would cause the beam of light 109 to no longer be perpendicular to the vertical-horizontal plane in this example, the shape and form of the second bar 102 may be such that it safely supports the apparatus 100 from tilting or dislocating. This may be accomplished by providing the second bar 102 with a flat bottom side with a large surface area and, optionally, being relatively heavy, to provide a good support when the apparatus 100 is placed horizontally on a flat surface, such as the floor of a room or a table. Also, the second bar 102 may be provided with separate means to secure the apparatus 100 to the surface on which it is placed, such as screws, straps, magnets, adhesive means or a holder of the kind commonly used to secure table lamps to a table.

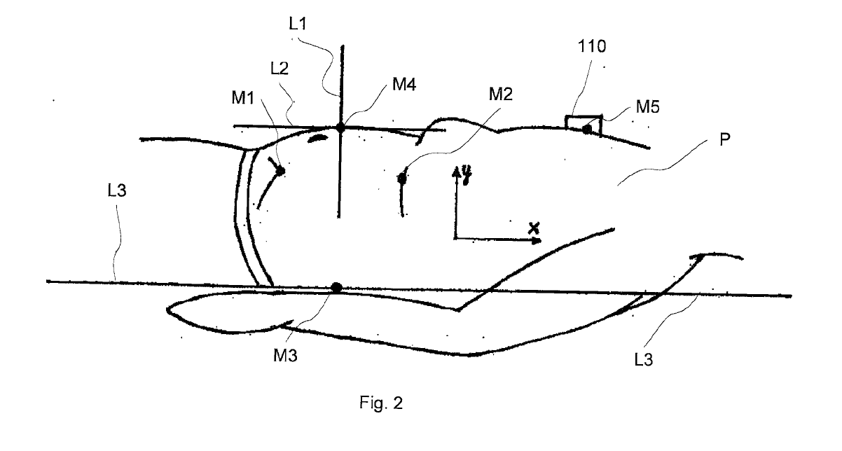

According to one embodiment of the invention, the apparatus 100 may be positioned in such a direction to, and at such a distance from, a person P, that when the beam of light 109 is exposing the same, or an immediately nearby extension unit 110 (see FIG. 2), the resulting reflected light is clearly visibly detectable by a user of the apparatus 100, or detectable by other suitable means, such as by a photodetector well known in the art.

The photodetector, employed for the detection of reflected light from a person P or extension unit 110 above, may be a camera such as a WEB-camera.

The distance between the light emitting device 104 of the apparatus 100 and the person P during measurements is preferably 0.01 to 10 m, and even more preferred 0.01 to 2 m.

Examples of said immediately nearby extension unit 110 include a piece of material, such as wood, plastic or metal, that is no larger in size or weight than it can be placed on a person P without unacceptable discomfort for the same. Extension unit 110 serves as an extension of a persons P body by allowing reach of the beam of light 109 when being placed on parts of the body that is not readily reachable by the beam of light 109 in the position that the person P is placed. Extension unit 110 may, for example, be placed on the breastbone of a person P lying horizontally on the back with the beam of light 109 coming directly from either the left or the right side, i.e. perpendicular to the sagittal plane of the body. Extension unit 110 may furthermore be provided with one or several light sensing means, which may be selectively sensitive to the light emitted from the light emitting device 104 so that a signal is transmitted from the object upon exposure of that light. Light sensing means, selectively triggered to transmit a signal by light of a particular wavelength, is well known in the art. The signal may be a radio-signal that can be intercepted by a receiver, such as a receiver integrated in or with a computer or control unit 111, or a sound-signal from electronics optionally integrated with the object, that could be heard by a user of the apparatus 100, all well known in the art. The signal may also be a fluorescent light readily seen by a user of the apparatus 100 and due to a coating of the object with a fluorescent material well known in the art.

According to one embodiment, the light emitting device 104, from which the beam of light 109 is coming, is preferably a laser, for example, of the same type as employed in pen-lasers used in public presentations, or a suitable aiming laser commonly used for firearms that may be purchased from, for example, Laser Devices Inc. The light emitting device 104 may also be, either by selection of such mode by a user or constantly, a laser in which the emitted beam of light 109 is periodically changing direction in the x- or the y-plane, i.e. the plane of the second bar 102 and the first bar 101, respectively, with such a rate that user of the apparatus 100 gets the visual impression of a line on the object, such as a person P, which the beam hits. The user is thereby guided in the identification of which parts of the object are located along the current e.g. x- or y-direction.

In order to make e.g. the visual impression possible, the light emitting device 104 may alternatively emit a sheet of light generated by, in the light emitting device 104, integrated optics and/or one or several lenses as well known in the art, that spread an initially generated beam of light into a sheet of light formed as the letter “V”. The sheet of light is optionally initiated by selection of the user by switching between different modes of the light emitting device 104, such as “beam mode” and “sheet mode”. The sheet of light is generating the visual impression of a line on the object, such as preferably in the coronal or transverse plane of a person P, or on extension unit 110, which the sheet of light hits.

According to one embodiment as partly illustrated in FIGS. 1 and 2, during measurements, a person P, such as a child or adolescent, may be placed lying down horizontally on the back in front of the apparatus 100, preferably in a comfortable way, such as being placed on a suitable bed or table with a flat surface, with an imaginary line along the spinal cord being essentially parallel to the x-axis, i.e. the second bar 102, and the same imaginary line being essentially perpendicular to the z-axis, i.e. the direction of the beam of light 109.

During measurements, a person P may also be standing up on the feet in an upright horizontal direction with the apparatus arranged so that the beam of light 109 or the center axis of the corresponding sheet of light, i.e. the z-direction of the apparatus, is perpendicular to e.g. the sagittal or coronal plane of the body of person P.

During measurements, a person P, such as a child or adolescent, is however preferably lying down horizontally on the back as this position is highly comfortable and the person P may even sleep.

The placement of a person P and the apparatus 100 may occur in any order of convenience and the person P is preferably naked or dressed in tight thin clothing on the parts of the body from which coordinates are to be collected.

A user of the apparatus 100, which may be a nurse, doctor, physician, teacher, parent or anyone else with a desire to collect data of relevance for a person's P status with regard to body fat mass, may independently and as desired move the first bar 101 and the first sliding member 103 to position the beam of light 109, or the center of the same, at any x- and y-coordinate within the limits defined by the effective length of the second and first bars 102, 101, which is preferably such that a person P may be exposed at any part of the body, and even more preferred at the part of the body in between the neck and the upper part of the thigh, by the beam of light 109.

When a desirable relative placement of a person P and the apparatus 100 has been arranged in accordance with the description above, such as the arrangement as illustrated in FIG. 1, the user of the apparatus 100 may select a first x-position of the person's P body by moving, simultaneously or one at a time, the first bar 101 and the first sliding member 103 until the reflection of the beam of light 109, which may be visually detected by the user as a spot or line on the person's P body, corresponds to the first x-position. The first bar 101 may then be secured at this first x-position by first connecting means 105 and its relative placement on the second bar 102, i.e. the corresponding x-coordinate of the person P, may be noted or by other means registered by the user.

When the first bar 101 is secured at the first x-position, a user may move the first sliding member 103 from a position where the reflection of the beam of light 109 from the person's P body is detectable, to a position where the same reflection from the body no longer is detectable, or the beam of light 109 is hitting an object behind the body, or the reflection distinctly changes character as is occurring when the beam of light 109 misses the body completely or hits it at a low angle, respectively. The beam of light 109 may also move away from the body and reach extension unit 110. At that point, a user may note or register the relative placement of the first sliding member 103 on the first bar 101, equivalent to the “limiting y-coordinate” of person P, at the selected first x-position, equivalent to the “coherent x-coordinate” of person P for said limiting y-coordinate.

When the beam of light 109 misses the body of the person P, the detection of the beam of light 109 with the same type of light sensing means selectively sensitive to the light emitted from the light emitting device 104, as herein before mentioned, may aid the user in the selection of x- and y-positions. For example, a suitable screen or wall, positioned on the opposite side of person P in relation to apparatus 100 and in the direction of the beam of light 109, may be provided with said light sensing means and thus transmit an indicative signal when the beam of light 109 hits the same. Preferably, extension unit 110 and the screen or wall are provided with different light sensing means to allow for easy differentiation of the occurrences of the beam of light 109 hitting the former or the latter.

Thereafter, a second x-position may be selected, and the same procedure as conducted for the first x-position may be repeated in order to collect the corresponding limiting y-coordinates for the second x-position, i.e. second coherent x-coordinate. Any number of x-positions, i.e. coherent x-coordinates, may accordingly be selected for the collection of the corresponding limiting y-coordinate(s). Generally the selection of, and the determination of the corresponding limiting y-coordinate(s) at, two different x-positions, i.e. corresponding coherent x-coordinates, is preferred.

The first sliding member 103 may be moved by a user in two directions, i.e. toward the second bar 102 or away from the same, to allow for the registration of, for example, two limiting y-coordinates of a person P at any selected x-position, i.e. coherent x-coordinate. One limiting y-coordinate may correspond to the intersection between the persons P body and the surface on which the person P is placed, such as lying down on the back, and constitutes a useful vertical reference line L3 in the case said surface is horizontal, or to the lower ending of the person's P body in the case this is not in contact with the underlying surface.

In addition, a skilled user of the apparatus 100, may accordingly collect x-y-coordinates corresponding to different parts of a person's P body, such as the highest point of the hipbone M1 and the lowest point of the ribs M2, and x-y-coordinates M5 corresponding to the extension unit 110, which may represent the height of the breastbone from a vertical reference line.

Different data sets, each consisting of one or several of limiting y-coordinate(s) together with the respective coherent x-coordinate(s), as well as x-y-coordinate(s) of different body part(s) and/or extension unit 110 and/or the y-coordinate corresponding to said vertical reference line, may be collected in accordance with the description above by different arrangements of a person P in relation to the apparatus 100. For example, the person P may be placed horizontally with the back facing downwards so that one of the person's P sides is facing the apparatus 100, i.e. a sagittal view, or with the side downward so that the person's P front is facing the apparatus 100, i.e. a coronal view. These arrangements would, upon the collection of data according to the description above, result in two data sets, one corresponding to a sagitall projection of a person P lying horizontally on the back and one corresponding to a coronal projection of a person P lying horizontally on the side, respectively.

During collection of the data sets, the beam of light 109 can advantageously not be sensed by the person P, and the beam of light 109 is preferably of a non penetrable wavelength, thus opposing a minimal risk of any health hazard to the person P.

In addition, x-y-coordinates may be collected accordingly in, for example, the sagittal and in the coronal plane of a person A who is standing up vertically, by arranging the apparatus 100 with the second bar 102 horizontally, the first bar 101 vertically, and with the beam of light 109 directed towards the person P.

Data sets, as described and collected according to the description and method above by employment of the apparatus 100, generated at different time-points, such as time-point with an interval in between them from several days to a few month, may be further mathematically manipulated and analyzed by the one skilled in the art and are valuable for the generation of metrics or anthropometric measurements and for the monitoring over time of the health status, with regard to e.g. body fat mass, of a person P.

According to one embodiment of the invention, there is provided a control unit 111 connected to the apparatus 100, into which a user may register data, or from which a user may read data from the apparatus 100, such as the x-y-coordinates of the beam of light 109 or data from any related sensor(s), such as light sensing means, or by which a user may initiate calculations on stored data and, optionally, via which a user may control the apparatus 100. The control unit 111 may store data from the apparatus 100, such as data from the means for x-, y- or z-position determination and perform calculations based on the same, such as calculating indexes and/or metrics known in the art. The control unit 111 enables a user of the apparatus 100 to read output, such as the present x-position of the first bar 101, the present y-position of the first sliding member 103, and calculated indexes or metrics, and store information of relevance, such as patient records comprising patient data and related two-dimensional coordinates, e.g. x- y- and x-z-coordinates, and calculated indexes and/or metrics. The patient data may comprise, for example, the name, the address, the weight and height, medical history and date of birth of a person P. A user of the apparatus 100 may optionally control the positioning of the first bar 101 and the first sliding member 103 via the control unit 111 as the same may be in control of devices known in the art, such as servo-motors, that automatically moves the first bar 101 and the first sliding member 103 to positions as desired by the user. A user of the apparatus may optionally control the mode of the light emitting device 104, or the corresponding, via the control unit 111, e.g. switching between “beam mode” and “sheet mode”, as desired. The control unit may be a personal computer (PC), provided with suitable software and hardware for input/output, or any other suitable control unit as well known in the art.

According to one embodiment of the invention, there is provided an apparatus 100 for the collection of two-dimensional coordinates of a growing child or adolescent lying down horizontally on the back, said coordinates being coordinates in the sagittal plane of the body.

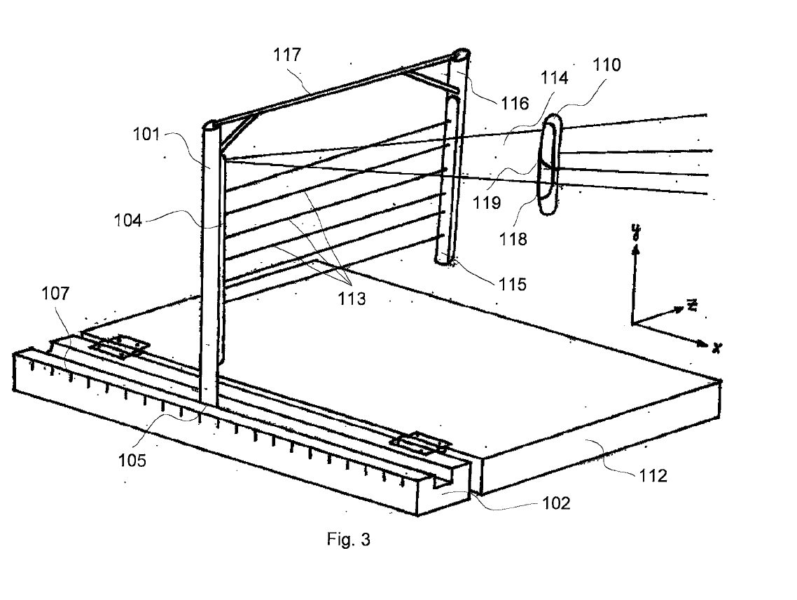

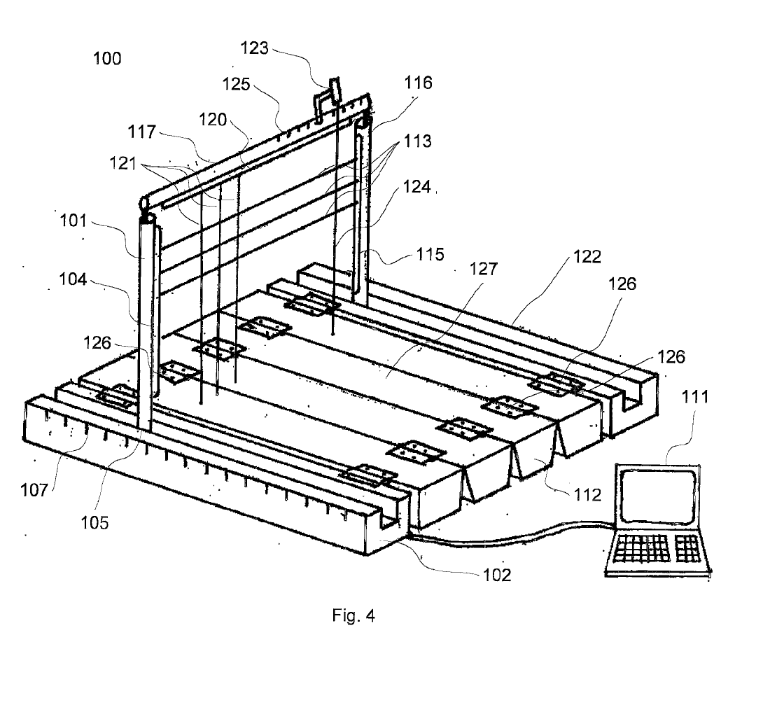

According to one embodiment of the invention as illustrated in FIGS. 3 and 4, the apparatus 100 is provided with an integrated bench 112 that may be stationary in, or (un)foldable into, the x-z-plane, i.e. the plane perpendicular to the plane defined by the first bar 101 and the second bar 102. The integrated bench 112 has a flat upper surface located at, either permanently or when (un)folded into, a predetermined y-coordinate corresponding to reference line L3, whereby a user of the apparatus does not need to separately determine the y-coordinate of reference line L3 as this is known. The integrated bench 112 may be connected to the second bar 102 by suitable connecting means known in the art, such as hinge joints 126. Means for facilitating the folding and unfolding of the integrated bench 112 are known in the art. For example, the integrated bench 112 may consist of separate parts that are connected with hinge joints 126 in a suitable way known in the art, whereby a flat comfortable surface in the x-z-plane is formed as the bench is (un)folded by a user who is setting up the apparatus for measurement of two-dimensional coordinates of a person P. When the integrated bench 112 is set for use of the apparatus 100 with the first and second bars 101, 102 arranged in the vertical and horizontal direction, respectively, i.e. being (un)folded into the x-z-plane, its upper surface is preferably located at a level below any beam of light of the apparatus 100 traveling in the z-direction, or within the lower 10% of the y-direction of the total possible range of such beams. The integrated bench 112 is preferably of a type which allows the apparatus 100 to be foldable into a format, such as “briefcase format”, whereby the apparatus 100 is highly movable and can be easily transported between different locations for measurements.

According to one embodiment of the invention as illustrated in FIGS. 2, 3 and 4, the light emitting device 104 may emit a plurality of beams and/or sheets of light, preferably a plurality of beams of light 113 and one sheet of light 114. Hence, the light emitting device 104 may comprise a plurality of light emitting sources, preferably lasers, located at a short distance, such as 0.1 to 5 mm, from each other. The light emitting sources may be placed on a row in the y-direction, i.e. in the same direction as the first bar 101, and the distance from the first to the last one in this row is preferably greater than the distance between M4 or M5, and L3, i.e. the greatest vertical distance in the sagittal plane, of a person P lying on the back on a horizontal flat bench. The light emitting device 104 may optionally be positioned directly onto the first bar 101 (as illustrated in FIGS. 3 and 4), i.e. without the need for a connection to a first sliding member 103.

According to one embodiment of the invention, light sensing means 115 may consist of an array of photo electric sensors, i.e. a plurality of photo electric sensors, preferably selectively sensitive to the light emitted by light emitting device 104, as known in the art. The photo electric sensors are preferably placed within a distance of 0.1 to 5 mm from each other in a row in the y-direction and may be of an integrated type as known in the art.

According to one embodiment of the invention, light sensing means 115 may consist of an array of photo electric sensors, i.e. a plurality of photo electric sensors, synchronized by suitable means known in the art, such as mechanically or electrically, with the plurality of beams of light 113, whereby each and one of these sensors is capable of detecting illumination caused by the corresponding single beam of light, coming from the corresponding light source which is placed right in front of it. Upon such detection of illumination, every single sensor may be capable of generating a positive light indication signal. Such signals and their practice are well known in the art and include, for example, an analog electric signal that may be converted to a digital signal (e.g. “1” of “1” or “0”), which may be further transmitted as input to a computer or to control unit 111, to allow for automatic calculation of y-coordinates. For example, should a single given beam of light of the plurality of beams of light 113 reach the corresponding sensor of the plurality of photo electric sensors, a digital “1” is generated. If, however, this single beam of light does not reach the corresponding sensor, as in the case of interruption by the body of a person P, the corresponding digital “0” is generated.

According to one embodiment of the invention, light sensing means 115 consists of one single photo electric sensor, whereby the illumination of the light sensing means 115 with one or several of the plurality of beams of light 113 is resulting in a positive light indication signal.

According to one embodiment as illustrated in FIGS. 3 and 4, the apparatus 100 is provided with a connecting bar 117, extending in the z-direction, which connects the first and third bar 101, 116 to keep these parallel in the y-z-plane independent of the relative position of the first bar 101. The connecting bar 117 is preferably removable and replaceable to allow for easy assembly of the apparatus 100 when a person P is placed in its immediate vicinity or, for example, on an integrated bench 112 of the same, for measurements.

According to one embodiment, the connecting bar 117 is provided with a second light emitting device, 120 or 123, from which one or several beams of light are emitted in the y-direction and towards a person P.

According to one embodiment, the second light emitting device is a single laser 123 that is movable along the connecting bar 117 and emits a single beam of light 124. Furthermore, the single laser 123 or the connecting bar 117 may be provided with means for relative z-position determination as known in the art, such as a scale 125 on the connecting bar 117.

According to one embodiment, the second light emitting device is constituted by a plurality of lasers 120, located at a distance of 0.1 to 5 mm from each other, and placed in a row on and along the connecting bar 117, and emitting a plurality of beams of light 121.

According to one embodiment as illustrated in FIG. 4, the integrated bench 112 is provided with laser selective sensing means 127, whereby the light emitted from the second light emitting device, 120 or 123, is detectable. Upon illumination of the laser selective sensing means 127 with one or several of the plurality of beams of light 121, or with the single beam of light 124, laser selective sensing means 127 may result in a positive light indication signal, in analogy to the positive light indication signal of light sensing means 115, which may be further transmitted as input to a computer or to control unit 111. The laser selective sensing means 127 are preferable covering the entire surface of the integrated bench 112 on the same side as a person P is placed, whereby the area of the bench outside the area of the sagittal plane of the body of a person P is covered by laser selective sensing means 127. Hence, laser selective sensing means 127 may detect the closest beam of light, relative the body, from the second light emitting device, 120 or 123, which is outside the area of the sagittal plane of the body of a person P. Suitable laser selective sensing means 127 are known in the art and include any suitable photosensitive detector, that may consist of an array of photosensitive semiconductors capable of generating an electric output signal upon illumination, that is sheet-formed and preferably foldable to allow facile placement on the integrated bench 112, in combination with a light filtering sheet, in order to allow only light of the same wavelength as the light emitted from the second light emitting device, 120 or 123, to reach the photosensitive detector.

According to one embodiment, the control unit 111 may (a) read input in the form of one or several positive light indication signal(s) from the light sensing means 115, or from the laser selective sensing means 127, (b) transmit output signals in the form of signals to the light emitting device 104, or to the plurality of lasers 120, or to the single laser 123, whereby each and one of the from any of said device or laser(s) emitted beam(s) of light individually can be turned on or off in accordance with an algorithm stored in control unit 111.

According to one embodiment, the apparatus 100 comprises (a) a plurality of lasers 120, emitting a plurality of beams of light 121, (b) a light emitting device 104, emitting a plurality of beams of light 113, (c) light sensing means 115 consisting one single photo electric sensor, capable of transmitting a positive light indication signal to control unit 111, (d) laser selective sensing means 127, capable of transmitting a positive light indication signal to control unit 111, and (e) control unit 111, wherein a suitable algorithm for control and registration and calculation of data of/from the apparatus 100 is stored.

Hence, after having placed a person P horizontally, in accordance with the description herein, and so that the coronal plane of the body is encompassed by the plurality of beams of light 121 and, at least, the upper part of the sagittal plane of the body is encompassed by the plurality of beams of light 113, two dimensional coordinates of person P may be collected, in the sagittal and coronal plane, at the present x-position of the first bar 101 by: (i) Turning on, each and one and one at a time, the beams of the plurality of beams of light 121, (ii) Registering the presence or absence of a positive light indication signal from the laser selective sensing means 127 for each beam of light during the time-period it is turned on, (iii) calculating the distance, corresponding to the coronal width of the body, from the first to the last beam of light associated with the absence of a positive light indication signal, (iv) Repeating “i” to “ii”, with the plurality of beams of light 113 in combination with light sensing means 115, for an estimation of the upper y-coordinate of the body in the sagittal plane, which corresponds to the uppermost beam of light associated with the absence of a positive light indication signal.

An additional set of two-dimensional coordinates may thereafter be collected at another x-position by moving the first bar 101 to a new position and repeating steps “i” to “iv”.

A user may thus conveniently collect two-dimensional coordinates from both the coronal and sagittal plane of the body of a person P, without the need of person P to move position or be touched in any way.

According to one embodiment of the invention, the apparatus 100 comprises a light emitting device 104 that emits a plurality of beams and/or sheets of light, as described herein above, and light sensing means 115 in the form of a row of a plurality of photo electric sensors in the y-direction, as described herein above. The plurality of beams of light 113 and the photo electric sensors are synchronized so that each beam is detected by a corresponding sensor, irrespective of the position of the first bar 101, provided that no object, such as a person P, is hindering said beam or beams. Means for the synchronization include, for example, a third bar 116 arranged in the y-direction, onto which the sensors are placed, which is mechanically connected, as known in the art, to the first bar 101 by, for example, a connecting bar 117. Limiting y-coordinates may thus be determined by registration, automatically by a control unit 111 or manually by a user, of e.g. the lowest detected beam of light (for an upper limiting y-coordinate) at the corresponding x-coordinate onto which the first bar 101 is placed at. The distance between the first bar 101 and the third bar 116 may be such that a person P can be placed, lying down horizontally on the back, easily between these bars, and preferably between 0.5 to 1.5 m.

According to one embodiment, the light emitting device 104 comprises a plurality of lasers located at a distance of 0.1 to 5 mm from each other, these lasers are placed in a row in the direction of the first bar 101 and emitting a plurality of beams of light 113 in a direction perpendicular to the plane defined by the first bar 101 and the second bar 102.

According to one embodiment, the apparatus 100 comprises a third bar 116 extending in the y-direction and in front of the beams of light 113. On the third bar 116 is located light sensing means 115 reachable by each and one of the plurality of beams of light 113. The third bar 116 may be placed in front of the beams of light 113 manually or by other means, such as being mechanically synchronized with the first bar 101 as known in the art.

According to one embodiment as illustrated in FIG. 4, the apparatus 100 is provided with a fourth bar 122. The movement of the third bar 116 in the x-direction may be facilitated by the aid of this fourth bar 122, which extends in the x-direction and is optionally integrated with the apparatus 100, via for example hinge joints 126 or other suitable means as known in the art. This fourth bar 122 acts as support by allowing the third bar 116 to slide therein by means known in the art, such as a low friction incision, and helps to keep the same in front of the beams of light 113 as the first and third bars 101, 116 are moved in the x-direction.

According to one embodiment of the invention, the apparatus 100 simultaneously comprises a light emitting device 104, which emits a plurality of beams and/or sheets of light and is placed on first bar 101, a third bar 116 provided with light sensing means 115 in the form of photo electric sensors, and an integrated foldable bench 112. The third bar 116 and the photo electric sensors being mechanically synchronized with the first bar 101 and the plurality of beams 113, respectively. The bench being foldable into the x-z-plane and configured, as known in the art, for comfortable placement of a person P lying on the back.

According to one embodiment of the invention, the light emitting device 104 is emitting a sheet of light 114 in the x-z-plane, and the extension unit 110 is shaped as a rod and provided with light sensing means 118 in the form of a row of a plurality of photo electric sensors, placed at a distance of preferably 0.1 to 5 mm from each other along extension unit 110. The sheet of light is directed toward a point just above the breastbone of a person P lying horizontally on the back on a flat surface and from whom two-dimensional coordinates are to be collected. The rod-shaped extension unit 110 is placed in a vertical direction on the breastbone of said person P, whereby the single sensor 119, of the plurality of photo electric sensors, which is located at the same y-coordinate as the sheet of light 114, is detecting the sheet of light 114. Accordingly, the distance from the reference line L3 to the top of the breastbone may be calculated based on the y-coordinate of the sheet of light 114 and the distance from the breastbone to the single sensor 119 detecting the sheet of light 114.

According to one embodiment of the invention, the rod-shaped extension unit 110 is provided with a wireless connection to control unit 111 and with a button, whereby a user may turn on and off the sheet of light 114 with the button, and a signal from the single sensor 119 may be transmitted to control unit 111 for subsequent identification of the same and calculation of the coordinates of e.g. the breastbone of person P. The user may thus not need to be near the control unit 111 for measurement of the coordinates of e.g. the breastbone, but only have to activate the button on extension unit 110.

According to one embodiment of the invention, apparatus 100 may be placed above, such as hanging from the ceiling, a person P lying horizontally on the back on a flat surface, whereby the first bar 101 is positioned horizontally, the second bar 102 is positioned horizontally, and the beam of light 109 or the corresponding beams or sheet(s) of light are directed towards person P, hence permitting collection of two-dimensional coordinates of a person P from the front side, i.e. in the coronal plane, while the person P is lying down on the back.

According to one embodiment of the invention, there is provided a method for use of apparatus 100 for the estimation of the body fat mass of a person P, the method comprising (a) placing a person P on the back on a flat surface in the horizontal plane with a side of the body, i.e. sagittal plane, facing the apparatus 100, the person P being naked on the part of the body from the neck to the upper part of the thigh, (b) registering at least two limiting y-coordinates of the person's P body, and (c) using said limiting y-coordinates for the estimation of the body fat mass of the person P.

According to one embodiment of the invention, the light emitting device 104 of apparatus 100 is a laser, which optionally may be set to periodically change direction of the emitted beam of light 109 in the x-z-, or in the y-z-plane.

According to one embodiment of the invention, the light emitting device 104 has built in optics whereby the said device emits a sheet of light shaped as the letter “V”.

According to one embodiment of the invention, the y-position of the first sliding member 103, and the x-position of the first bar 101, is determined by visual reading of y- and x-scales 108, 107, respectively.

According to one embodiment, the apparatus 100 is provided with means for relative x-position determination 107 of the first bar 101 along the second bar 102, such as a scale imprinted on the second bar 102.

One embodiment of the invention is illustrated by the following example describing the collection of x-y-coordinates useful for the determination of metrics related to body fat mass as known in the art, such as the F-index (Flodmark index):

A person P, being a child or adolescent, is lying comfortably on the back on a horizontal flat bench, being naked on the upper part of the body above the hipbone. A user places the apparatus 100, in accordance with the description herein, so that the beam of light 109, being a laser, is perpendicularly directed toward the side of the person P, i.e. the sagittal plane of the body. The user then registers, in any order and in accordance to the description herein, (i) the x-y-coordinates (or alternatively only the x-coordinate thereof) of the upper part of the hipbone M1, (ii) the x-y-coordinates (or alternatively only the x-coordinate thereof) of the lower part of the ribs M2, (iii) the y-coordinate M3 (or alternatively any x-y-coordinate along the reference line L3) for reference line L3 corresponding to the intersection between the body and the bench, (iv) the x-y-coordinates (or alternatively only the y-coordinate thereof) of the uppermost point of the body M4 of person P along vertical line L1 being vertically extended from the middle of an imaginary line between M1 and M2, and (v) the x-y-coordinates M5 (or alternatively only the y-coordinate thereof) of extension unit 110 being placed on the breastbone of person P or of an equivalent position thereof. The vertical distance between M5 and L3 is related to the height of person P. The vertical distance between M4 and L3, being equivalent to the distance between the horizontal lines L2 and L3, is related both to the height and the body fat mass of person P. As known in the art, the relationship between the two distances is indicative of the relative body fat mass of person P at the time of measurement.

One embodiment of the invention is illustrated by the following example, describing the collection of x-y-coordinates useful for the determination of metrics related to body fat mass as known in the art: (i) placing the apparatus 100 with the first bar 101 extending in a vertical direction and the second and third bar 102, 116 extending in a horizontal direction, and the connecting bar 117 extending in a direction perpendicular to the plane defined by the first and second bars 101, 102, the connecting bar 117 being connected to the first and third bars 101, 116, the light emitted from the light emitting source 104 and plurality of lasers 120 or single laser 123 being directed towards the intended location of measurement of person P, (ii) placing the person P on the back on the integrated bench 112 arranged in the horizontal plane with the sagittal plane of the body facing and being parallel to the plane defined by the first bar 101 and the second bar 102, and the coronal plane of the body being perpendicularly reachable by the plurality of beams of light 121 or said single beam of light 124, and (iii) registering at least two limiting y-coordinates in the sagittal plane and at least two limiting z-coordinates in the coronal plane of person P.

According to one embodiment of the invention, apparatus 100 is provided with calculating means, such as calculating means of the control unit 111, and with optional automatic moving means, such as means integrated with the first connecting means 105, of first bar 101, whereby the x-coordinate corresponding to the middle point of two user selected x-coordinates, such as M1 and M2, is calculated and presented to the user, or to which the first bar 101 is automatically moved upon user selection of two x-coordinates, respectively. Said calculating means and said automatic moving means are well known in the art.

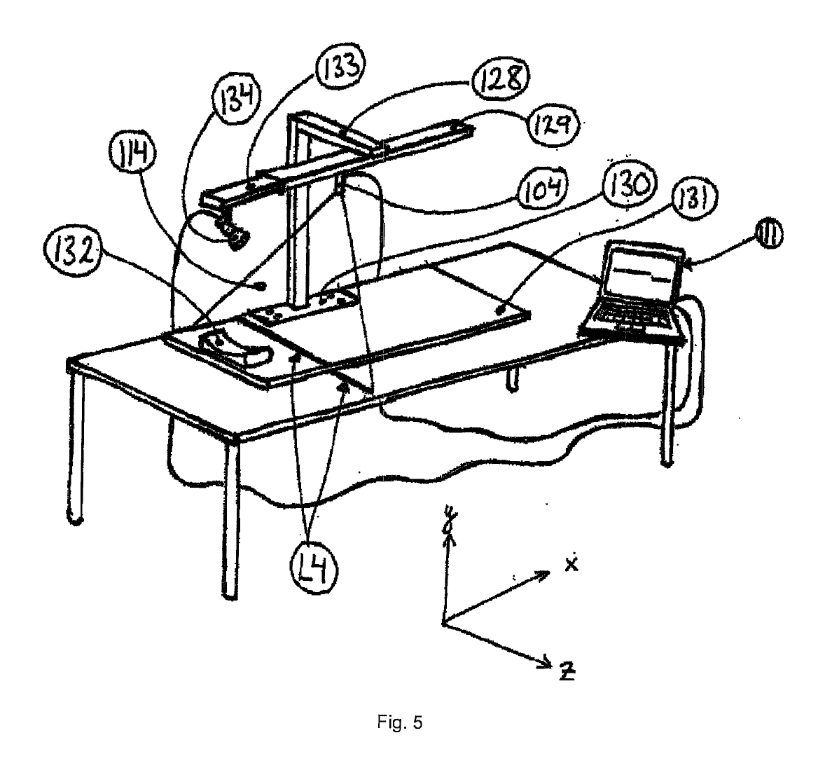

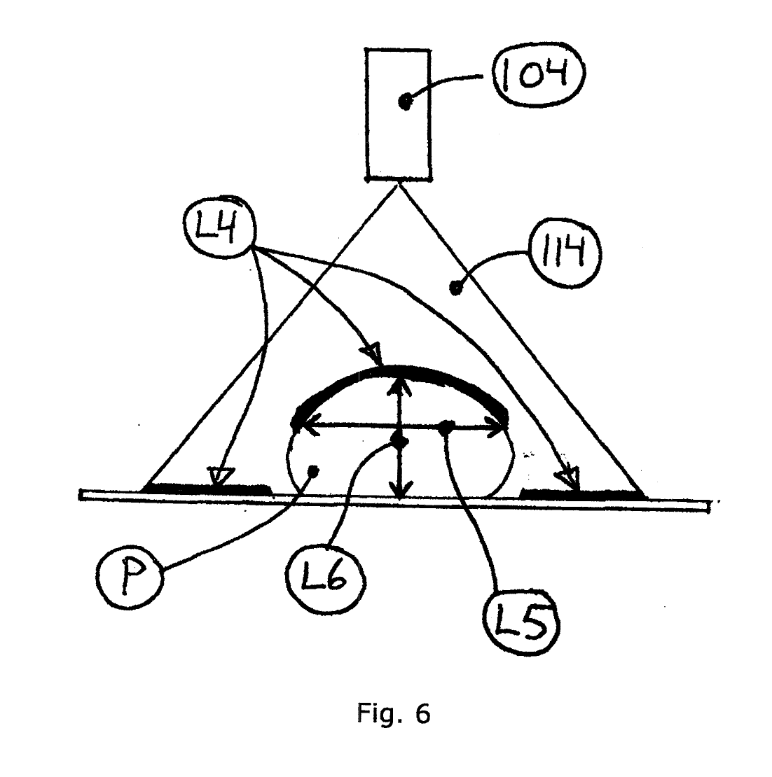

According to one embodiment illustrated in FIGS. 5 and 6, the apparatus 100 is comprising a supporting bar 128 arranged to provide spatial support for the placement of a hanging bar 129. The hanging bar 129 is placed in a direction essentially parallel to an imaginary line along the spinal cord of a person P while facing the front side of the person P. A preferred direction of the hanging bar 129, spatially supported in this way, is horizontally. The person P may then, during measurements, lay down comfortably in comparison to the more uncomfortable position of standing up vertically. Standing up vertically is, however, a potential applicable direction for measurements on the person P. The shape of the above described arranged supporting bar 128 may be “inverted L” (as shown in FIG. 5) or any other shape, such as straight angled or curved, which is suitable for holding the hanging bar 129 in front of the center of a person P.

The distance between the hanging bar 129 spatially supported in this way, or any part thereof facing the person P, may be 0.05 to 3 m, preferably 0.3 to 1.5 m.

The supporting bar 128 may be provided with fastening means 130 for fastening onto or an external surface, e.g. a bed plate 131. Hence, the supporting bar 128 is then not movable in relation to the bed plate 131 or a person P, when placed thereon upon measurements. Fastening means 130 include, for example, a metallic plate. The fastening means 130 may be e.g. welded, screwed or glued to the supporting bar 128. The fastening means may be provided with holes through which it might be fastened to the bed plate 131 by employment of screws or bolts. Many other types of suitable fastening means 130 are well known in the art.

The bed plate 131 is arranged to be placed horizontally in the x-z-plane on a suitable bed or table with a flat surface. When so arranged, an imaginary line along the spinal cord of a person P, placed comfortably on the bed plate 131, is essentially parallel to the x-axis. The bed plate 131 may be made of a suitable material such as glass-fibre laminate. The bed plate 131 may optionally be covered with a material or sheet which is comfortable for a person P placed thereon. This material or sheet is preferably easily cleaned to e.g. minimize the chances of spreading decease between different persons P. The bed plate 131 may optionally be provided with a head-rest pad 132. A person P might place the head or neck thereon in order to lay down comfortably in a horizontal position on the bed plate 131.

The second sliding member 133 is slidingly connected to the hanging bar 129 to allow a user to move the second sliding member 133 to a position on the hanging bar 129, i.e. in the x-direction, as desired by the user. This movement is only limited by the effective length of the hanging bar 129. When the second sliding member 133 has been moved to a desired x-position, manually or automatically, it is arranged to remain in that position until a user desires, either manually or by automatic means, to move it to another x-position. The second sliding member 133 is slidingly connected to the hanging bar 129 by means such as, for example, a tightening bolt, a natural friction element, or a fitting suitable for connecting a first bar to a second bar. The second sliding member 133 may be a straight bar which is fitted partly inside the hanging bar 129. The second sliding member 133 may also be any other shape or type which allows for connection of several objects, such as light emitting devices 104 and cameras 134, at a distance from each other onto the second sliding member 133 and along the direction of the hanging bar 129. These objects are arranged to face the person P during measurements and to follow the movement of the second sliding member 133.

One or several light emitting devices 104 are connected to the second sliding member 133 whereby they, at all times, follows the movements of the second sliding member 133. The light emitted from each of the light emitting devices 104 may be a sheet of light 114, traveling in the y-z-plane. One or several lines L4 are hence projected onto the surface of the body of a person P and onto the surface adjacent to and behind the body, such as the bed plate 131, during measurements. Each of the light emitting devices 104 are preferably arranged in such a way that the imaginary projection in the x-z-plane of each line L4 is essentially perpendicular to an imaginary line along the spinal cord of the person P independent of the position of the second sliding member 133 on the hanging bar 129.

One or several cameras 134 are connected to the second sliding member 133 whereby these, at all times, follows the movements of the second sliding member 133.

The resulting reflected light from the body of a person P and from the surface adjacent to the body, corresponding to the above mentioned projected lines L4, is detected by the one or several cameras 134. The one or several cameras 134 provide input data to control unit 111, which may be a PC. A camera 134 may not be arranged in the same plane as a sheet of light 114, but instead detecting a line L4 from an angle, such as 10 to 80°, relative the plane of the sheet of light 114, which is giving rise to the corresponding line L4. The projected line L4 may thus be detected by the camera 134 as a curvature, when projected onto a curved body, or as a straight line, when projected on a flat surface such as the bed plate 131.

The coronal and sagittal width, i.e. distances, related to the distances L5 and L6, respectively, of a person P in the y-z-plane at the x-coordinate were a sheet of light 114 is resulting in a projected line L4, may be calculated by suitable means. Such means include for example well known picture analysis of one or several pictures taken by the corresponding camera 134, by e.g. the control unit 111. This picture analysis may include the determination of the coordinates for the description of the line L4 as detected by a camera 134. By mathematically handling of the, by a camera 134, detected line L4, the distances L5 and L6 may be determined by taking other necessary input parameters into account, as known in the art. Input parameters for such picture analysis and mathematical handling include distances between each and one of a light emitting device 104, the corresponding camera 134, and the surface onto which the person P is placed. As dependant on different parameters, such as the distance of the light emitting device from the body of a person P, the coronal width will be slightly underestimated if approximated as the distance L5 unless an error correction factor is used as an additional input parameter. Such an error correction factor is preferably determined, as known in the art, for a particular setup of the apparatus 100 and used as an additional input parameter for the calculations.

The relative position of the second sliding member 133 on the hanging bar 129 may be determined by suitable means, such as an electronic position sensor, to provide input data to control unit 111. The relative positions of the one or several light emitting devices 104 and the one or several cameras 134 may be calculated, preferably by the control unit 111, from the relative position of the second sliding member 133 and the known arrangement of the former onto the second sliding member 133.

Preferably, the apparatus 100 is portable and easily dissembled and assembled so that a user easily may move it between various geographic locations for use.

Preferably, the components constituting the apparatus 100 are provided with smooth and comfortable surfaces which are easily cleaned in order to e.g. be comfortable for a user and a person P and to diminish the chances of spreading contagious diseases between different persons P.

Preferably, the apparatus 100 is provided with a back-up power source as known in the art, such a the battery of a portable PC or any other suitable battery, so that e.g. it might be used at locations where external power is not available.

According to one embodiment, the light emitting device is emitting light of a wavelength and intensity which is giving rice to a projected line L4 which is narrow in width and easily detected by a camera 134, such as a green laser. The light is preferably diffusing to a minimal extent in the skin which might otherwise give rice to broadening of the reflected line. One advantage of such an easily detected line L4 is that it is easier to recognize and separate the line L4 from potential back-ground interference during picture analysis and mathematical manipulation.

According to one embodiment, the second sliding member 133 is provided with one light emitting device 104 and one camera 134.

According to one embodiment, one of either a camera 134 or a light emitting device 104 is stationary fastened, such as e.g. on the hanging bar 129, and the other one is attached to the second sliding member 133 and thus movable together with the second sliding member 133.

According to one embodiment, a camera 134 is arranged further away from the person P than a light emitting device 104 so that it might be placed closer to the same in the x-direction while still allowing measurement of at least distances L5.

According to one embodiment, the distance between a camera 134 and a light emitting device is between 0.1 to 1.5 m, preferably between 0.3 to 1 m.

According to one embodiment, the camera 134 is a WEB-camera or any other suitable camera provided with digital output as known in the art.

One embodiment of the invention is illustrated by the following example describing the collection of data comprising the coronal and sagittal width, being related to the distances L5 and L6, respectively, at two different x-positions by use of an apparatus of the invention comprising one light emitting device 104 and one camera 134, as illustrated in FIG. 5, which projects a line L4 onto the body of a person P, as illustrated in FIG. 6:

A person P, being a child or adolescent, is lying comfortably on the back on the bed plate 131, being naked on the upper part of the body above the hipbone with his or her arms pointing straight out in the z-direction. (i) A user then places the second sliding member 133, e.g. by hand, so that the sheet of light 114, coming from the light emitting device 104, is drawing a line L4 which coincides with the navel (navel position). (ii) The user then registers the corresponding picture of the camera 134 with the control unit 111. Optionally, the user may mark this dataset with the text string “navel position” to indicate which x-position this dataset was collected at for future reference. (iii) The user then places the second sliding member 133 so that the sheet of light 114, coming from the light emitting device 104, is drawing a line L4 which coincides with the highest point of the breast bone (breast bone position). Optionally, the user may mark this dataset with the text string “breast bone position” to indicate which x-position this dataset was collected at for future reference. (iv) The user then registers the corresponding picture of the camera 134 with the control unit 111. (v) The user then employs the control unit 111 to calculate the coronal and sagittal width at each of the two x-positions (navel position and breast bone position) from the pictures that were registered with the camera 134 under “ii” and “iv” above.

According to one embodiment, x-positions from which data, such as the distances L5 and L6, might be collected include the highest or widest point of the abdomen, the highest point of the breastbone, the hipbone and the lowest point of the rib of e.g. a person P placed vertically on the back.

According to one embodiment, the apparatus 100 may be used to detect irregularities or abnormalities on or within the body of the person P. Such irregularities or abnormalities include enlarged organs, e.g. the liver, a cancer, or any other irregularities or abnormalities as known in the art.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. An apparatus for estimating body fat mass by using anthropometric measurements as a proxy measurement for the degree of obesity of a person by determination of the sagittal width or the coronal width at one or several positions of the person using the apparatus, the sagittal width or the coronal width being used for the generation of the anthropometric measurements, the apparatus comprising:
a hanging bar placed in a direction essentially parallel to an imaginary line along the spinal cord of the person and in front of the person;
a supporting bar configured to provide support for the spatial placement of the hanging bar, the supporting bar being provided with a fastener for fastening onto surface;
a sliding member being slidingly connected to the hanging bar to allow a user to move the sliding member to a position on the hanging bar as desired;
at least one light emitting device configured to emit a sheet of light shaped as the letter “V” directed toward the body of the person, whereby at least one line is projected onto the body of the person and simultaneously onto the surface adjacent to and behind the body of the person;
at least one camera configured to detect the at least one line; and
a control unit configured to accept data input from the at least one camera and to perform calculations based on the data input to provide at least one estimate of the sagittal width or the coronal width of the person;
wherein, at least one of the at least one light emitting device and the at least one camera is connected to the sliding member.

2. An apparatus for estimating body fat mass by using anthropometric measurements as a proxy measurement for the degree of obesity of a person by determination of two-dimensional coordinates in at least one plane of the person using the apparatus, the two-dimensional coordinates being used for the generation of the anthropometric measurements, the apparatus comprising:
a first bar and a second bar, the first bar configured to be movable along the second bar and the first bar being perpendicular to the second bar;
a first connector connecting the first bar to the second bar; and
a light emitting device configured to emit light in a direction perpendicular to a plane defined by the first bar and the second bar towards the person;
wherein one of
the light emitting device is connected to a first sliding member, and the first sliding member is connected to the first bar and movable along the first bar, and the light emitting device is configured to emit a beam of light, which reflection is visually detectable for enabling a user of the apparatus to determine x-coordinates and y-coordinates, and
the light emitting device is configured to emit a plurality of beams of light towards a light sensing mechanism in the form of at least one photo electric sensor; and
wherein the light emitting device is a laser emitting light which reflection is visually detectable.

3. The apparatus according to claim 2, wherein the light sensing mechanism is a row of a plurality of photo electric sensors in the direction of the first bar, synchronized so that each of the beams of light is detected by a corresponding photo electric sensor provided that the person is not hindering the beam of light.

4. The apparatus according to claim 2, wherein the light sensing mechanism is one single photo electric sensor, whereby the illumination of the light sensing mechanism with at least one of the plurality of beams of light results in a positive light indication signal.

5. The apparatus according to claim 2, further comprising:
means for relative x-position determination of the first bar along the second bar, and
means for relative y-position determination of the first sliding member, along the second bar.

6. The apparatus according to claim 2, wherein the light emitting device is one single laser or a plurality of lasers.

7. The apparatus according to claim 2, further comprising a control unit.

8. The apparatus according to claim 2, further comprising an integrated bench being stationary or foldable into a plane perpendicular to the plane defined by the first bar and the second bar.

9. The apparatus according to claim 2, further comprising the light sensing mechanism, which is in the form of a plurality of photo electric sensors; and
wherein the light emitting device being positioned directly onto the first bar and includes a plurality of light emitting sources that are located at a distance of 0.1 to 5 mm from each other, and the light emitting sources are placed in a row in the direction of the first bar and emitting the plurality of beams of light in a direction perpendicular to the plane defined by the first bar and the second bar, and the beams of light are directed towards the plurality of photo electric sensors.

10. The apparatus according to claim 2,
wherein the light emitting device includes a plurality of lasers located at a distance of 0.1 to 5 mm from each other, the lasers being placed in a row in the direction of the first bar and emitting the plurality of beams of light in a direction perpendicular to the plane defined by the first bar and the second bar; and
wherein the apparatus further comprises;
a third bar extending in the y-direction and in front of the plurality of beams of light,
the light sensing mechanism located on the third bar whereby being reachable by the plurality of beams of light,
a connecting bar extending in the z-direction and between the first bar and the third bar, whereby the first bar and the third bar are kept parallel and in the y-z-plane independent of the relative position of the first,
an integrated bench connected to the second bar, the integrated bench being stationary or foldable into a plane perpendicular to the plane defined by the first bar and the second bar,
a second light emitting device located on the connecting bar and including one of
(a) one single laser movable along the connecting bar and emitting a single beam of light, the connecting par provided with means for relative z-position determination (125), and
(b) a plurality of lasers located at a distance of 0.1 to 5 mm from each other, the plurality of lasers being placed in a row along the connecting bar and emitting a plurality of beams of light,
wherein the direction of the single beam of light or the plurality of beams of light parallel to the direction of the first bar and towards the person.

11. The apparatus according to claim 10, wherein the light sensing mechanism includes one of
(a) a plurality of photo electric sensors synchronized with the plurality of beams of light from the light emitting device whereby each of the photo electric sensors is configured to detect illumination caused by the corresponding beam of light resulting in an individual positive light indication signal, and
(b) one single photo electric sensor whereby the illumination of the light sensing mechanism with at least one of the plurality of beams of light from the light emitting device results in a positive light indication signal; and
wherein the apparatus further includes
laser selective sensing means provided on the integrated bench and configured to give a positive light indication signal upon illumination with at least one of the plurality of beams of light or the single beam of light from the second light emitting device, and
a control unit (111), configured to
(a) read input including at least one positive light indication signals from the light sensing mechanism or from the laser selective sensing means, and
(b) give output signals including signals to the light emitting device or the second light emitting device whereby the emitted beams of light individually can be turned on or off in accordance with an algorithm.

12. A system for estimating body fat mass and degree of obesity of a person by determination of two-dimensional coordinates, comprising:
an apparatus according to claim 2; and
an extension unit for placement on the person, such that the extension unit functions as an extension of the body of the person.

13. The system according to claim 12, wherein the extension unit is rod-shaped;
wherein the system further includes light sensing means in the form of a row of a plurality of photo electric sensors along the extension unit (110); and
wherein—the light emitting device is emitting a sheet of light configured to illuminate the extension unit when placed on a person.

14. A method for estimating body fat mass by using anthropometric measurements as a proxy measurement for the degree of obesity of a person by determination of the sagittal width or the coronal width at one or several positions of the person using the apparatus of claim 1, the sagittal width or the coronal width being used for the generation of the anthropometric measurements, the method comprising:
placing the person in front of the at least one light emitting device and the at least one camera;
collecting data from the at least one camera; and
performing calculations based on the data to provide an estimate of the sagittal width or the coronal width of the person.

15. A method for estimating body fat mass by using anthropometric measurements as a proxy measurement for the degree of obesity of a person, and by determination of two-dimensional coordinates from the sagittal plane or the coronal plane of the person, using the apparatus according to claim 2, the method comprising the steps of:
(i) placing the apparatus with the first bar extending in one of a vertical direction for sagittal determination and a horizontal direction for coronal determination, the second bar extending in the horizontal direction, and the light emitted from the light emitting source being directed towards the intended location of measurement of the person;
(ii) placing the person on the back on a flat surface in the horizontal plane with the sagittal plane or the coronal plane of the body facing and being parallel to the plane defined by the first bar and the second bar of the apparatus; and
(iii) registering at least two limiting y-coordinates of the person.

16. A method for estimating body fat mass by using anthropometric measurements as a proxy measurement for the degree of obesity of a person, and by determination of two-dimensional coordinates from the coronal plane and the sagittal plane of the person, using the apparatus according to claim 10, the method comprising:
(i) placing the apparatus with the first bar and the third bar extending in a vertical direction, the second bar extending in a horizontal direction, the connecting bar extending in a direction perpendicular to the plane defined by the first and the second bar and being connected to the first bar and the third bar, and the light emitted from the light emitting device and the second light emitting device being directed towards the intended location of measurement of the person;
(ii) placing the person on the back on the integrated bench arranged in the horizontal plane with the sagittal plane of the body facing and being parallel to the plane defined by the first bar and the second bar and the coronal plane of the body being perpendicularly reachable by the light from the second light emitting device; and
(iii) registering at least two limiting y-coordinates in the sagittal plane and at least two limiting z-coordinates in the coronal plane of the person.

17. A method for estimating body fat mass by determining two-dimensional coordinates in the sagittal plane of a person, from which the body fat mass is subsequently estimated, using the apparatus according to claim 2, the method comprising:
placing the person on the back on a flat surface in the horizontal plane;
registering the x,y-coordinates of the upper part of the hipbone of the person;
registering the x,y-coordinates of the lower part of the ribs of the person;
registering the y-coordinate of a horizontal reference line the reference line corresponding to the intersection between the body of the person and the flat surface;
registering the y-coordinate of the uppermost point of the body of the person along a vertical line being vertically extended from the middle of an imaginary line between the upper part of the hipbone and the lower part of the ribs;
registering the y-coordinate of the top of the breastbone of the person;
calculating the vertical distance between the uppermost point of the body and the horizontal reference line; and
calculating the vertical distance between the top of the breastbone and the horizontal reference line.
